(19) United States
(12) Patent Application Publication
(10) Pub. No.: US 2008/0274956 A1
(43) Pub. Date: Nov. 6, 2008

(54) Fusion Protein Comprising a Bh3-Domain of a Bh3-Only Protein

(75) Inventors: Christophe Bonny, Lausanne (CH); Didier Coquoz, St. Sulpice (CH)

Correspondence Address: SUGHRUE MION, PLLC, 2100 PENNSYLVANIA AVENUE, N.W., SUITE 800, WASHINGTON, DC, 20037, US

(73) Assignee: XIGEN S.A., Lausanne (CH)

(21) Appl. No.: 11/720,413

(22) PCT Filed: Nov. 18, 2005
(86) PCT No.: PCT/EP05/12389

§ 371 Date: Apr. 11, 2008

(30) Foreign Application Priority Data
Nov. 29, 2004 (EP) 04028278.2

(51) Int. Cl. A61K 38/17; C07K 7/00; A61K 38/08; A61P 35/00; C07K 14/47

(52) U.S. Cl. 514/12; 530/328; 530/350; 514/15

(57) ABSTRACT

This invention relates to a fusion protein comprising at least one first portion (I) comprising a trafficking sequence and at least one second portion (II) comprising a full-length or partial BH3-domain sequence of a BH3-only protein, said fusion protein comprising D-enantiomeric amino acids in retro-inverso order in its portion (I). Furthermore, the invention relates to pharmaceutical compositions containing said fusion protein as well as to the use of said fusion protein.

This invention relates to fusion proteins comprising at least one first portion (I) comprising a trafficking sequence and at least one second portion (II) comprising a full-length or partial BH3-domain sequence of a BH3-only protein, said fusion protein comprising D-enantiomeric amino acids in retro-inverso order. Furthermore, the invention relates to pharmaceutical compositions comprising said fusion proteins as well as to the use of said fusion proteins.

The cells of multicellular organisms are members of a highly organized community. The number of cells in this community is tightly regulated, not only by controlling the rate of cell division, but also by controlling the rate of cell death. If cells are no longer needed, they commit suicide by activating an intracellular death program. This significant process is called apoptosis. Apoptosis is also known as programmed cell death and is signalled by a cascade of proteins. Changes, e.g. mutations of proteins involved, that prevent this normal programmed cell death, play an essential role in many diseases, in particular cancer. Therefore, cancerous cell growth often results from defective control of cell death. Moreover, resistance to apoptosis is a key characteristic of malignant cells enabling them to increase in number and survive.

The mitochondria-dependent pathway for apoptosis is governed by Bcl-2 family proteins. Bcl-2, Bcl-xL, Bak, and many other members of the Bcl-2 family have a hydrophobic stretch of amino-acids near their carboxyl-terminus that anchors them in the outer mitochondrial membrane. In contrast, other Bcl-2 family members such as Bid, Bim, and Bad lack these membrane-anchoring domains, but target to mitochondria in response to specific stimuli. Still others have the membrane-anchoring domain, but keep it latched against the body of the protein, until stimulated to expose it (e.g. Bax).

Bcl-2 family proteins are conserved throughout metazoan evolution, with homologs found in vertebrate and invertebrate animal species. Several types of animal viruses also harbour Bcl-2 family genes within their genomes, including herpes simplex viruses implicated in cancer, such as the Epstein-Barr Virus (EBV) and Kaposi sarcoma herpesvirus (KSHV). Both pro-apoptotic and antiapoptotic Bcl-2 family proteins have been delineated. The relative ratios of anti- and pro-apoptotic Bcl-2 family proteins dictate the ultimate sensitivity or resistance of cells to various apoptotic stimuli, including growth factor deprivation, hypoxia, radiation, anticancer drugs, oxidants, and Ca²⁺ overload.

During apoptosis Bcl-2 family proteins especially regulate (promote or inhibit) the release of proteins, such as cytochrom c, Smac/DIABLO, apoptosis inducing factor, HtrA2 and endonuclease G, from mitochondria. Certain Bcl-2 family proteins (pro-apoptotic members) are major regulators of the commitment to programmed cell death as well as executioners of death signals at the mitochondrion. In summary, the Bcl-2 family consists of pro-apoptotic members triggering mitochondrial protein release and anti-apoptotic members inhibiting mitochondrial protein release. Moreover, the family can be divided into three main subclasses [(a), (b) and (c)]. Subclasses (a) and (b) are probably similar in structure to the pore-forming domains of bacterial toxins, such as the colicins and diphtheria toxin. These helical pore-like proteins include both anti-apoptotic proteins (subclass (a)) as well as multidomain pro-apoptotic proteins (subclass (b)). The anti-apoptotic members (a), e.g., Bcl-2 and Bcl-xL, display conservation throughout all four Bcl-2 Homology (By domains (BH1-4, also called “multidomain anti-apoptotic proteins”). Some of the proaptotic members (b), e.g., Bax and Bak, possess sequence homology in BH1-3 domains (BH1-3, also called “multidomain pro-apoptotic proteins”). In contrast, some of the proaptotic members of subclass (c), e.g., Bid, Noxa, Puma, Bik, Bim, Bad, Bmf, DP5/Hrk and Bok possess sequence homology in only one domain, namely the alpha-helical BH3 domain (called “BH3-only proteins”). The BH3 domain typically consists of an amphipathic a-helix of about 15 to 20 amino-acids length, typically 16 amino acids.

BH3-only proteins (subclass (c)) act as afferent effectors of various pro-apoptotic and anti-apoptotic signals (Hunt A. et al (2001) Science 293, 1784-1785). All BH3-only proteins have the ability to bind to and to functionally antagonize anti-apoptotic Bcl-2 family proteins (Hunt A. et al., supra). Their BH3 domain inserts into a hydrophobic crevice on the surface of antiapoptotic proteins such as Bcl-X_{L}. They transduce multiple death signals to the mitochondrion by interacting with anti-apoptotic Bcl-2 family proteins and inducing apoptosis by a mechanism that requires the presence of at least one of the multidomain pro-apoptotic Bcl-2 family proteins Bax or Bak. Moreover, some but not all BH3-only proteins have the ability to bind to multidomain pro-apoptotic members Bax or Bak.

BH3-only protein Bid, for example, can promote the pro-apoptotic assembly and function of Bax and Bak by itself, whereas other BH3-only proteins do not function as such. By using short peptides representing the alpha-helical BH3 domain of BH3-only protein Bid, it was shown that said “Bid-peptides” are capable of inducing oligomerization of Bax and Bak to release cytochrom c. This property is attributed to BH3-only protein Bim (Korsmeyer S. J. et al (2000) Cell Death Differ 7, 1166-1173). In summary, Bid binds (multidomain) pro-apoptotic Bax and Bak as well as (multidomain) anti-apoptotic Bcl-2 and Bcl-xL and is capable to activate Bax and Bak directly.

In contrast, peptides representing BH3 domains of BH3-only proteins Bad or Bik lack the ability to directly activate Bax and Bak, but retain the ability to bind to anti-apoptotic Bcl-2 family proteins (Letai A et al (2002) Cancer Cell 2, 183-192). This is also shown by another study using peptides derived from BH3 domain of BH3-only protein Bad which stimulate Bax activity only in the presence of Bcl-xL (Moreau P.-F. et al (2003) The Journal of Biological Chemistry, vol. 278, no. 21, 19426-19435). Thus, BH3 domains of BH3-only proteins possess distinct functions. A “Bid-like” BH3 domain “activates” pro-apoptotic Bax and Bak, whereas a “Bad-like” BH3 domain “sensitizes” pro-apoptotic Bax and Bak by occupying the pocket of anti-apoptotic Bcl-2 family members. In summary, BH3 domains of BH3-only proteins represent two classes of tool compounds that initiate cell death using genetically defined pathways (Letai A et al. (2002) Cancer Cell 2, 183-192).

As described above, BH3-only proteins were characterized in various studies and their function and mechanism of action was analysed. The results indicate that their BH3 domains may efficiently induce apoptosis in cells expressing high levels of anti-apoptotic Bcl-2 family members by triggering the release of active multidomain pro-apoptotic proteins from said anti-apoptotic proteins.

Thus, BH3 domains of BH3-only proteins are an interesting subject and form an efficient tool in the treatment of various diseases caused by defective control of programmed cell death. However, prior art does not suggest a way to directly transport said proteins or their BH3 domains, respectively, to a desired localisation in an organism or a cell. One of the main problems is to permeabilize the inner membrane of cells. Several studies include the initial polyarginine transduction approach (Wang et al, (2000), Cancer Res. 60, 1498-1502; Holinger et al. (1999), J. Biol. Chem. 274, 13298-13304). However, various amphipathic α-helical peptides, especially cationic peptides, can be attracted to negatively charged membranes, including mitochondrial membranes, where they can non-specifically disrupt the lipid matrix and membrane barrier function (Matsuzaki (2001) Biochem. Soc. Trans. 29, 598-601; Ellerby et al (1999), Nat. Med. 5, 1032-1038). Another approach using the construct ANT-BH3BAD, combining antennepedia (ANT) as internalization moiety and their BH3 domain of BAD, was toxic. However, toxicity was not correlated with the Bcl-2 pathway (Vieira et al. (2002), Oncogene 21, 1963-1977; Schimmer et al. (2001), Cell Death Differ. 8, 725-733).

Therefore, it is an object of the present invention to provide a system capable of directly transporting a BH3 domain of a BH3-only protein to a desired localisation in an organism.

According to the invention a fusion protein is provided which comprises at least one first portion (I) comprising a trafficking sequence and at least one second portion (II) comprising a partial or full-length BH3-domain sequence of a BH3-only protein, said fusion protein comprising D-enantiomeric amino acids in retro-inverso order in its portion (I) and, optionally, (II).

The fusion protein of the invention comprises (at least in its portion (I)) D-enantiomeric amino acids in retro-inverso order (also called “retro-inverso” protein or “retro-inverso” fusion protein). “Retro-inverso” relates to an isomer of a linear peptide in which the direction of the regular or native L-amino acid sequence is reversed and the chirality of each amino acid residue (due to the chirality of the D-amino acids in contrast to the naturally occurring L-amino acids) is inverted. The terms “fusion protein” and “protein” according to the invention are intended to have the same meaning. Retro-inverso fusion proteins according to the invention can be constructed, e.g., by synthesizing a reverse of the amino acid sequence for the corresponding native L-amino acid sequence. In D-retro-inverso enantiomeric (fusion) proteins the positions of carbonyl and amino groups in each single amide bond are inverted, thereby ensuring that the position of the side-chain groups at each alpha carbon is preserved, if compared with regular L-amino acid peptide sequences. Retro-inverso fusion proteins of the invention possess a variety of useful properties. For example, they enter cells more efficiently and are more stable (especially in vivo) and show lower immunogenicity than corresponding L-amino acid fusion proteins. Since naturally-occurring proteins contain L-amino acids, almost all decomposing enzymes, like proteases or peptidases, cleave peptide bonds between adjacent L-amino acids. Consequently, proteins composed of D-enantiomeric amino acids in retro-inverso form are largely resistant to proteolytic breakdown. Evidently, the retention time in cell is increased for fusion proteins of the invention as compared to their regular non-inverted L-amino acid analogues.

Portion (I) of the inventive fusion protein serves as a carrier or trafficking sequence. A “trafficking or carrier sequence” (as a portion of the inventive fusion protein) is any sequence of amino acids that directs a fusion protein or portion (II) of the fusion protein, respectively, into the cell cytoplasm or, even further, to a specific cellular destination. The trafficking sequence can e.g. direct the fusion protein to a desired location within the cell, e.g., the nucleus, the ribosome, the endoplasmatic reticulum, a lysosome, or a peroxisome. Consequently, in a preferred embodiment the trafficking sequence of the fusion protein of the invention directs the fusion protein to a defined cellular location. Anyhow, the trafficking sequence can direct the inventive fusion protein across the plasma membrane, e.g., from the extracellular cell environment through the plasma membrane into the cytoplasma thereby enhancing the cellular uptake of the fusion protein or its drug portion (II) (cargo portion), respectively, in particular by enhancing its cell permeability or by enhancing its intracellular retention time.

In general, a functionally effective portion (I) of the inventive fusion protein which comprises said trafficking sequence is defined as an amino acid sequence comprising at least 4 (contiguous) D-amino acids. Functionally effective amino sequences as comprised by portion (I) should show strong basic properties. Preferably, said functionally effective amino acid sequences contain at least 60%, preferably at least 65%, more preferably at least 70%, even more preferably at least 75%, even more preferably at least 80%, yet more preferably at least 85%, yet more preferably at least 90%, most preferably at least 96% basic amino acids, preferably arginine and/or lysine. Functionally effective portions (I) of the inventive fusion protein preferably comprise an amino acid sequence ranging from 4 to 50 D-amino acids, more preferably from 4 to 40 D-amino acids, even more preferably from 4 to 30 D-amino acids, even more preferably from 4 to about 20 D-amino acids and most preferably from 4 to about 10 D-amino acids.

Preferably, a trafficking sequence according to the invention can be derived, e.g., from a known membrane-translocating sequence. In a particularly preferred embodiment of the invention, the trafficking sequence of the fusion protein of the invention is a D-enantiomeric amino acid sequence in retro-inverso order of a TAT protein of human immunodeficiency virus (HIV). TAT is a viral protein indispensable for the HIV infection cycle. This protein is described in, e.g., WO 94/04686, U.S. Pat. Nos. 5,804,604 and 5,674,980, each incorporated herein by reference. Portion (I) of the fusion protein of the invention is linked to a portion (II), whereby portion (I) comprises some or all of the 86 amino acids of the Tat full-length sequence, however, in its retro-inverso D-form. In particular, a functionally effective portion (I) of an inventive fusion protein that has fewer than 86 amino acids can be used, which still exhibits improved cell uptake activity and, optionally, has additional localization information for entry into the cell nucleus. Tat sequence segments responsible for mediating entry and uptake into cells can be defined using known techniques (see, e.g., Franked et al., Proc. Natl. Acad. Sci, USA 86: 7397-7401 (1989). Preferably, portion (I) of the fusion protein of the invention comprises the basic region (amino acids 48-57 or 49-57, respectively of TAT and does not comprise TAT's cysteine-rich region (amino acids 22-36) as well as the exon 2-encoded carboxy-terminal domain (amino acids 73-86) of the naturally-occurring TAT protein. The reverse of Tat's basic region (aa 57 to 48 or 57 to 49, composed of D-amino acids) may be used as portion (I) or part of portion (I) of an inventive fusion protein. Sequences containing the reverse of Tat's basic region or consisting of the reverse are herein referred to as D-Tat sequence.

Therefore, a preferred functionally effective portion (I) of the inventive fusion protein comprises a D-TAT sequence, e.g. NH₂-RRRQRRKKRG-COOH or NH₂-RRRQRRKKR-COOH (both of which retro-inverso sequences of the above-defined naturally occurring Tat basic region), or comprises sequence variants according to generic D-TAT formula NH₂-XXXAXXXXX-COOH, wherein “X” relates to amino acid arginine or lysine and “A” relates to any non-basic amino acid. “A” is preferably located at position 4 of the above-given general formula. However, “A” may alternatively be positioned at any other intrasequential position, i.e. at position 2, 3, 5, 6, 7, or 8 instead of position 4. Moreover, the above-given general formula may be modified introducing additional non-basic residues by substituting one to four basic residues of the general formula with non-basic residues. In accordance with the above, portion (I) sequences are exclusively composed of D-amino acids in order to exhibit the same structural side chain pattern as the corresponding non-inverted sequences composed of L-amino acids.

In a particularly preferred embodiment of the invention portion I comprises trafficking sequences with the amino acid of FIG. 1A or FIG. 1B.

In another embodiment functionally effective portions (I) of the inventive fusion protein comprise a generic amino acid sequence of the following general formulae: NH₂-AmXAoXAn-COOH, NH₂-AmXXXAn-COOH, NH₂-AmXXAXAn-COOH, NH₂-AmXAXXAn-COOH, or NH₂-XAAX-COOH, wherein “X” relates to amino acid arginine or lysine, “A” relates to any non-basic amino acid, “m” is an integer from zero to fourteen, “n” is an integer, independent from m, between zero and fourteen and “o” is an integer, independent from m and n, between zero and five. It has to be noted that the general formulae of the above trafficking sequences, according to the invention, are exclusively composed of D-enantiomeric amino acids.

Specific examples for functionally effective portions (I) of the inventive fusion protein contain or may consist of the following sequences showing strong basic properties: NH₂-KTRR-COOH, NH₂-RLKR-COOH, NH₂-KPRR-COOH, NH₂-KRFQR-COOH; NH₂-GRIRR-COOH, NH₂-NIGRRRN-COOH, NH₂-RAGRNGR-COOH, NH₂-RPRR-COOH, NH₂-GKRRG-COOH, NH₂-KRRE-COOH, NH₂-RQKRGGS-COOH, NH₂-RKSR-COOH, NH₂-RGSRR-COOH, NH₂-RRQK-COOH, NH₂-RARKG-COOH, NH₂-RGRK-COOH, NH₂-RRRLS-COOH, NH₂-RPRRLSP-COOH, NH₂-RGRKY-COOH, NH₂-RPKRGMG-COOH, NH₂-GVRRR-COOH, NH₂-GYKKVGFSR-COOH, NH₂-KFSRLSK-COOH, NH₂-RRVR-COOH, NH₂-RRSRP-COOH, NH₂-RRRM-COOH, NH₂-KSMALTRKGGY-COOH, NH₂-RSRRG-COOH, NH₂-KMNPLPY-COOH. It has to be noted that that the above trafficking sequences, according to the invention, are exclusively composed of D-enantiomeric amino acids.

Portion (I) may comprise just one (i.e., continuous) basic cell membrane translocation sequence (D-retro-inverso form) analogous to the corresponding (naturally-occurring) translocation sequence, e.g. a D-Tat sequence as disclosed above. Alternatively, portion (I) may comprise two or more amino acid sequences in retro-inverso order which correspond to identical or different translocation sequences with strong basic properties. These retro-inverso D-form translocation motifs are synthesized on the basis of naturally-occurring protein(s) by using D-amino acids and by reverting its amino acid sequence order. In other words, an inventive fusion protein may comprise e.g. in its portion (I) a combination of two or more translocation sequences all of which are synthesized as retro-inverso D-form sequences. Typically, this combination of translocation motifs does not occur in any native protein sequence (as L-form). These two or more translocation sequences of portion (I) may be linked together with or without a linker sequence. If a linker sequence is desired, its length will preferably range from 2 to 20 amino acids.

As disclosed above, portion (I) may contain (one or more) D-enantiomeric amino acid translocation motifs (in retro-inverso order as compared to the native sequence), which are provided on the basis of its corresponding naturally-occurring L-form analog. Alternatively, portion (I) may contain D amino acid strings (in retro-inverso order) of L-form sequences, which are sequentially modified compared to the naturally occurring protein sequences (also defined as “derivatives” herein). These portion (I) derivatives (being provided in retro-inverso D-form, in other words the derivative D-form is an analog of the derivative L-form) typically possess carrier properties with uptake activity into the cell (or even, preferably, into the cell nucleus) that is substantially similar to that of the corresponding naturally-occurring protein, even though their sequence is not identical with the naturally occurring protein sequence.

In addition, sugar moieties and/or lipid moieties, e.g. cholesterol or other lipids, may be added to the inventive fusion protein, in particular to portion (I) of the inventive fusion protein or, eventually to any other portion, in order to further increase the membrane solubility of the fusion protein, e.g. to one or both termini of portion (I) to provide local lipophilicity at one or both termini. Alternatively or additionally, sugar or lipid moieties may be linked to the amino acid side chains, in particular side chains having hydroxyl and amino groups.

Portion (I) of the inventive fusion protein has to retain its cell permeability and/or its intracellular retention function. However, other functions may be added by modifications introduced into portion (I). Therefore, portion (I) can be modified, e.g., to efficiently direct the inventive fusion protein of the invention to a particular intracellular target localization. Correspondingly, portion (I) is modified such that a specific intracellular localization is awarded to portion (I) without loss of its enhanced cell permeability properties. Typically, a routing sequence for targeting the inventive fusion protein to specific cell compartments (e.g., endoplasmic reticulum, mitochondrion, gloom apparatus, lysosomal vesicles) can be introduced into portion (I). On the other hand, specific sequences which ensure cytoplasmatic localization of the inventive fusion protein may be added. E.g., portion (I) may comprise at least one further sequence which binds to one or more cytoplasmatic structure(s) in order to retain the fusion protein of the invention in the cytoplasm. Alternatively, alteration of the basic region thought to be important for nuclear localization (see, e.g., Dang and Lee (1989), J. Biol. Chem. 264:18019-18023; Hauber et al. (1989), J. Virol. 63:1181-1187; Ruben et al. (1989), J. Virol. 63:1-8) can result in a cytoplasmic location or partially cytoplasmic location of portion (I), and, therefore, of the fusion protein of the invention. Therefore, portion (I) may contain altered nuclear localization signals, which lead to cytoplasmatic localization of the inventive fusion protein.

As described above, BH3-only proteins are members of the Bcl-2 family representing regulators of apoptosis by interacting with other members of Bcl-2 family. Portion (II) of the inventive fusion protein is a amino acid sequence comprising one or more partial or full-length BH3-domain sequence(s) of a BH3-only protein (defined as a subclass of the Bcl-2 family proteins). Portion (II) may either comprise a D-retro-inverso form of the naturally occurring (full-length or partial) BH3-domain sequence, thereby ensuring that its D-amino acid side chains are positioned as in the native all-L-amino acid sequence, or, alternatively, may comprise the native all-L-amino acid sequence, both of which induce apoptosis by either interacting with at least one Bcl-2 family protein or by activating or sensitising at least one pro-apoptotic member of the Bcl-2 family.

If portion (II) of the inventive fusion protein comprises a partial BH3-domain sequence, it should have (in order to be a functionally effective) at least 4 (contiguous) D- or L-amino acids in either native or retro-inverso order (reflecting the respective fragment of at least 4 L-amino acids of a BH3-domain of a BH3-only protein). In a preferred embodiment portion (II) comprises exclusively D-amino acids. Preferred functionally effective portions (II) of the inventive fusion protein comprise a full-length or partial BH3-domain sequence composed of D-amino acids in retro-inverso order ranging from 4 to 20 D-amino acids, more preferably from 4 to 18 D-amino acids, more preferably from 4 to 15 D-amino acids, even more preferably from 4 to 13 D-amino acids, more preferably from 4 to about 10 D-amino acids, and yet more preferably from 4 to 8 D-amino acids. If partial BH3-domain sequences are used as portion (II) or part of portion (II), they have to remain functional, i.a. they are expected to retain their pro-apoptotic activity. Their functional activity can be tested by suitable assay methods, e.g. by binding assays (binding of portion II of the inventive fusion protein or of the inventive fusion protein itself to its native binding partner) or by assaying its pro-apoptotic activity by apoptosis assays.

Preferably, portion (II) comprises a partial or full-length of BH3-domain sequence of a BH3-only protein selected from the group consisting of Bid, Bad, Noxa, Puma, Bim, Bik, Bmf, DP5/Hrk and Bok either in its native L-form or in its retro-inverso D-form. The (full-length or partial BH3-sequences may be selected from e.g. any mammalian BH3-only protein, in particular from the human isoforms. Accordingly, portion (II) of the inventive fusion protein comprises the amino acid sequence of FIG. 2A, 2B, 2C, 2D, 2E or 2F (either in its native form composed of L-amino acids as shown in FIGS. 2A to 2F or in its retro-inverso D-form (composed of D amino acids), which means that the sequence of FIG. 2A to 2F have to be inverted by reverting the termini: native C-terminus is the N-terminus of the inverted form and the native N-terminus is the C-Terminus of the inverted form). Portion (II) of the inventive fusion protein may contain fragments of the native full-length BH3-only protein sequence (either in its L- or D-form), e.g. fragments of less than 50, preferably of less than 40 and even more preferably of less than 30 amino acids. These fragments of the native sequences typically comprise or at least partially (at least 7 amino acids of the BH3-domain sequence) comprise a BH3-domain sequence.

The sequence of portion (II) of the inventive fusion protein may contain one or more partial or full-length BH3-domain sequence(s) of BH3-only proteins either in its/their native L-amino acid form or in its/their inverted form being composed of D-amino acids. If portion (II) is composed of L-amino acids, it should be synthesized either by recombinant methods known to the skilled person or by peptide synthesis methods, e.g. solid phase synthesis. The L-amino acid sequence of portion (II) is afterwards coupled (by a second step) to the D-amino acid sequence of portion (I). Alternatively, chemical synthesis may allow to directly synthesize (without introducing a second coupling step) an inventive fusion protein composed of D-amino acids in its portion (I) and of L-amino acids in its portion (II). If, however, portion (II) is composed of D-amino acids as well, the fusion protein is chemically synthesized (according to the same methods as used for the synthesis of L-amino acid sequences) by using D-enantiomeric amino acids in retro-inverso order in order to invert the naturally occurring partial or full-length BH3-domain sequence and the e.g. naturally occurring trafficking sequence, e.g. the naturally occurring basic Tat-sequence.

The amino acid sequence of naturally-occurring BH3-only proteins or their fragments containing the BH3-domain and, correspondingly, their retro-inverso analogs composed of D-enantiomeric amino acids as part of portion (II) can be modified, for example, by addition, deletion and/or substitution of at least one amino acid of the naturally-occurring protein, to provide modified non-native sequences. Modifications, in particular conservative modifications, may be desired to e.g. modify the binding properties of portion (II) or modify its stability, e.g. increasing or decreasing its stability. Portion (II) with a modified primary sequence (as compared to naturally occurring BH3-domain or BH3-only protein sequences) may be produced by using the same techniques as for the synthesis of unmodified L-amino acid or D-amino acid sequences.

Fusion proteins of the invention are composed at least of portion (I) and portion (II). Moreover, the fusion protein according to the invention may comprise further portions (III), (IV), (V). These optional additional portions may award additional functions to the inventive fusion protein. The at least one further portion can be an amino acid, oligopeptide or polypeptide or an small organo-chemical compound and can be linked to the fusion protein of the invention at a suitable position, for example, the N-terminus, the C-terminus or internally coupled to amino acid side chains. Further portions (e.g., HA, HSV-Tag, His6) may render the inventive fusion protein amenable to purification and/or isolation. If desired, the fusion partner can then be removed from fusion protein of the invention (e.g., by proteolytic cleavage or other methods known in the art) at the end of the production process. Alternatively, further trafficking sequences for specific cell compartments or other functional sequences may be fused to the inventive molecule by adding an additional portion or may be incorporated into portion (I) or (II). Preferably, an additional portion (e.g. portion (III)) allows the inventive fusion protein to specifically bind to a certain cell type, e.g immune cells, hepatocytes, in particular tumor cells of organ, e.g. lymphocytes, neurons etc. Specificity is achieved by fusing ligands (e.g. ligands for extracellular portions of membrane proteins, like receptors, or anti-bodies directed to extracellular portions of membrane proteins, tumor cell markers), which bind to these cell markers, to the inventive fusion protein. Thereby, the inventive fusion protein may be directed specifically to target cells or tissues of an animal to be treated.

In another preferred embodiment, more than one, preferably two or three identical or non-identical, partial or full-length BH3-domain sequences may be incorporated into the inventive fusion protein either as part of portion (II) or as additional portion (e.g. (III)). In particular, partial or full-length BH3-domain sequences of different BH3-only proteins may be combined within the inventive fusion protein, e.g. Bid and Bad, Bim and Bad, Bik and Bad, Puma and Bad, Noxa and Bad, Bmf and Bad, DP5/Hrk and Bad, Bok and Bad, Bik and Bim, Bik and Bid, Bik and Puma, Bik and Noxa, Bik and Bmf, Bik and DP5/Hrk, Bik and Bok Bid and Puma, Bid and Noxa, Bid and Bim, Bid and Bmf, Bid and DP5/Hrk, Bid and Bok, Bim and Noxa, Bim and Puma, Bim and Bmf, Bim and DP5/Hrk, Bim and Bok, Puma and Noxa, Puma and Bmf, Puma and DP5/Hrk, Puma and Bok, Noxa and Bmf, Noxa and DP5/Hrk and Noxa and Bok. Preferably, if more an inventive fusion protein has more than one BH3-domain, the inventive fusion protein comprises a (partial or full-length) BH3-domain of a “Bid”-like and a “Bad”-like BH3-only protein. Typically, if more than one BH3-domain is present in the inventive fusion protein, both or all of them will either exhibit the retro-inverso D-form or the regular native L-form. Alternatively, an inventive fusion protein may combine D- and L-form BH3-domains.

Consequently, the fusion protein according to the invention typically has a length ranging from at least 12 to about 200 amino acids or more (dependent on the additional portions and/or the length of portion (II)), preferably from 12 to about 150 amino acids, more preferably about 20 to about 100 amino acids, even more preferably about 20 to about 75 amino acids, even more preferably about 20 to about 50 amino acids. If portion (II) is composed of L-amino acids, the fusion protein according to the invention preferably has a sequence according to FIG. 3A, 3B, 3C, 3D, 3E or 3F. Alternatively, the fusion protein may have a trafficking sequence (being contained by portion (I)) as shown in these figures and a fragment (being contained by portion (I)) of typically less than 50 contiguous amino acids of the full-length BH3-only protein sequences shown in these figures, the fragment comprising at least partially the BH3-domain sequence of an BH3-only protein. In another embodiment of the invention, the sequences of each portion (II) shown in these figures may be composed of D-amino acids and are reversed by reversing the termini resulting in all-D-amino acid retro-inverso sequences of portion (II).

In an preferred embodiment of the invention, the at least one first portion (I) and the at least one second portion (II) of the fusion protein of the invention are linked by a covalent bond. “Covalent bond” relates to a stable chemical link between two atoms produced by sharing one or more pairs of electrons. If present, further portions (III), (IV), (V) etc., as mentioned above, can also be linked by a covalent bond either to portion (I) or to portion (II).

In general, portion (I) and portion (II) can be coupled via a linker or directly (without linker) by an amide bridge formed between N-terminal and C-terminal, respectively, amino acids of portion (I) and (II). The C-terminal amino acid of portion (I) may be linked to the N-terminal amino acid of portion (II) or the N-terminal amino acid of portion (I) may be linked to the G terminal amino acid of portion (II). Thus, fusion proteins of the invention may have for example, a C-terminal or N-terminal trafficking sequence (portion (I)) depending upon which terminus of portion (I) is covalently linked to portion (II), whereby a linker may be added between portion (I), e.g., a sequence comprising a D-TAT sequence, and portion (II) as partial or full-length BH3-domain sequence containing portion.

If present, further portions (III), (IV), X etc., as mentioned above, can be coupled in an analogous manner with portion (I) or portion (II) or, optionally with each other or. Linker sequences can also be used to fuse the fusion protein of the invention with at least one other moiety/moieties as described below. Optional portions (III), (IV) etc. may be linked to the inventive fusion protein via the side chains of its D amino acids or may be attached to either terminus of the portion (I) or (II). The linkage via a side chain will preferably be based on side chain amino or hydroxyl groups, e.g. via an ester or ether linkage. It has to be noted that, according to the invention, all amino acids (of any of portions (I), (II), (III), (IV), (V) etc.) are of D-enantiomeric amino acids, which mimic its eventually naturally occurring analogue by being linked in retro-inverso order.

If linker sequences are used to fuse portion (I) and (II) or to fuse another portion, e.g. (III) to portion (I) and/or (II), the linker sequences preferably form a flexible sequence of 5 to 50 residues, more preferably 5 to 15 residues. In a preferred embodiment the linker sequence (either an all-D- or all-L-amino acid sequence) contains at least 20%, more preferably at least 40% and even more preferably at least 50% Gly residues. Appropriate linker sequences can be easily selected and prepared by a person skilled in the art.

Portion (I) and portion (II) can also be linked by chemical coupling in any suitable manner known in the art. However, attention is drawn to the fact that many known chemical cross-linking methods are non-specific, i.e., they do not direct the point of coupling to any particular site on the transport protein (or polypeptide) or cargo moiety. Thus, the use of non-specific cross-linking agents may attack functional sites or sterically block active sites, rendering the fused proteins biologically inactive. If present, further portions (III), (V), (V) etc., as mentioned above, can be coupled in an analogous manner to one another or to portion (I) and/or (II).

Coupling specificity can be increased by directly chemical coupling to a functional group found only once or a few times in one or both of the proteins/peptides/polypeptides to be cross-linked. An example is cystein which is the only amino acid containing a thiol group and which occurs only a few times in many proteins. Also, for example, if a protein/peptide/polypeptide contains no lysine residues, a cross-linking reagent specific for primary amines will be selective for the amino terminus of that protein/peptide/polypeptide. However, successful utilization of this approach to increase coupling specificity requires that the protein/peptide/polypeptide have the suitably rare and reactive residues in areas of the molecule that may be altered without loss of the molecule's biological activity.

Cysteine residues may be replaced when they occur in parts of a protein/peptide/polypeptide sequence where their participation in a cross-linking reaction would otherwise likely interfere with biological activity. When a cysteine residue is replaced, it is typically desirable to minimize resulting changes in protein (or polypeptide) folding. Changes in protein/peptide/polypeptide folding are minimized when the replacement is chemically and sterically similar to cysteine. Therefore, serine is preferred as a replacement for cysteine. However, a cysteine residue is introduced, introduction at or near the N- or C-terminus is preferred. Conventional methods are available for such amino acid sequence modifications, whether the protein/peptide/polypeptide of interest is produced by chemical synthesis or expression of recombinant DNA.

Coupling of the two constituents, e.g. for coupling the D-form of portion (I) to an portion (II) L-form, can be accomplished via a coupling or conjugating agent. There are several intermolecular cross-linking reagents which can be utilized, see for example, Means and Feeney, Chemical Modification of Proteins, Holden-Day, 1974, pp. 39-43. Among these reagents are, for example, N-succinimidyl 3-(2-pyridyldithio)propionate (SPDP) or N,N′-(1,3-phenylene)bismaleimide; N,N′-ethylene-bis-(iodoacetamide) or other such reagent having 6 to 11 carbon methylene bridges; and 1,5-difluoro-2,4-dinitrobenzene. Other cross-linking reagents useful for this purpose include: p,p′-difluoro-m,m′-dinitrodiphenylsulfone; dimethyl adipimidate; phenol-1,4-disulfonylchloride; hexamethylenediisocyanate or diisothiocyanate, or azophenyl-p-diisocyanate; glutaraldehyde and disdiazobenzidine. Cross-linking reagents may be homobifunctional, i.e., having two functional groups that undergo the same reaction. A preferred homobifunctional cross-linking reagent is bismaleimidohexane (BMH). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible cross-linking of proteins (or polypeptides) that contain cysteine residues. Cross-linking reagents may also be heterobifunctional. Heterobifunctional cross-linking agents have two different functional groups, for example an amine-reactive group and a thiol-reactive group, that will cross-link two proteins having free amines and thiols, respectively. Examples of heterobifunctional cross-linking agents are succinimidyl 4-(N-maleimidomethyl)cyclohexane-1-carboxylate (SMCC), m-maleimidobenzoyl-N-hydroxysuccimide ester (MBS), and succinimide 4-(p-maleimidophenyl)butyrate (SMPB), an extended chain analog of MBS. The succinimidyl group of these cross-linkers reacts with a primary amine, and the thiol-reactive maleimide forms a covalent bond with the thiol of a cysteine residue. Because cross-linking reagents often have low solubility in water, a hydrophilic moiety, such as a sulfonate group, may be added to the cross-linking reagent to improve its water solubility. Sulfo-MBS and sulfo-SMCC are examples of cross-linking reagents modified for water solubility. Many cross-linking reagents yield a conjugate that is essentially non-cleavable under cellular conditions. Therefore, some cross-linking reagents contain a covalent bond, such as a disulfide, that is cleavable under cellular conditions. For example, Traut's reagent, dithiobis(succinimidylpropionate) (DSP), and N-succinimidyl 3-(2-pyridyldithio)propionate (SPDP) are well-known cleavable cross-linkers. The use of a cleavable cross-linking reagent permits the cargo moiety to separate from the transport polypeptide after delivery into the target cell. For this purpose, direct disulfide linkage may also be useful. Chemical cross-linking may also include the use of spacer arms. Spacer arms provide intramolecular flexibility or adjust intramolecular distances between conjugated moieties and thereby may help preserve biological activity. A spacer arm may be in the form of a protein (or polypeptide) moiety that includes spacer amino acids, e.g. proline. Alternatively, a spacer arm may be part of the cross-linking reagent, such as in “long-chain SPDP” (Pierce Chem. Co., Rockford, Ill., cat. No. 21651H). Numerous cross-linking reagents, including the ones discussed above, are commercially available. Detailed instructions for their use are readily available from the commercial suppliers. A general reference on protein cross-linking and conjugate preparation is: Wong, Chemistry of Protein Conjugation and Cross-Linking, CRC Press (1991).

The present invention also encompasses fusion proteins, which mimic native functional sequences or its D-form analog, but are sequentially modified. These modified variants are defined as derivatives and are also fusion proteins of the invention. A “derivative” may comprise a derivative of portion (I) and/or portion (II) as well as portion (III), portion (IV), portion (V) etc. (if present) of the naturally occurring L-sequence or its corresponding retro-inverso D-form. It is intended to indicate a fusion protein which is derived from the native L-sequence (or its D-form analog) by substitution of one or more L- or D-amino acids at one, two or more of sites of the amino acid sequence, deletion of one or more amino acids at either or both ends of the inventive amino acid sequence or at one or more sites of the amino acid sequence, or insertion of one or more amino acids at one or more sites of the inventive amino acid sequence retaining its characteristic activity. Preferably, substitution of amino acid(s) relates to conservative substitution. Conservative substitutions typically include substitutions within the following classes: glycine and alanine; valine, isoleucine and leucine; aspartic acid and glutamic acid; asparagine and glutamine; serine and threonine; lysine and arginine; and phenylalanine and tyrosine. Thus, preferred conservative substitution groups are aspartate-glutamate; asparagine-glutamine; valine-leucine-isoleucine; alanine-valine; phenylalanine-tyrosine and lysine-arginine. By these sequence alterations of portion (I) and/or (II) (as well as portion (III), (IV), (V) etc., if present), e.g. the stability and/or effectiveness of the inventive fusion proteins can be modified, e.g. enhanced. Fusion proteins of the invention being modified as compared to their native L-form (or to its D-form analog; both L- and D-form reflecting the native sequence are also termed “parent sequences”) have to remain homologous, e.g. in sequence, in function, and in antigenic character or other characteristics, with a protein having the corresponding parent sequence. It is particularly preferred that the derivatives of the native trafficking sequence being comprised in portion (I) remain functional (maintain its character as cell permeable moiety), whereas the derivatives of the native (partial or full-length) BH3-domain sequence being comprised in portion (II) maintain their pro-apoptotic property. These inventive fusion proteins derived from a native parent sequence possess altered properties being advantageous over the properties of the parent sequence (e.g. increased pH optimum, increased temperature stability etc.). It is understood that e.g. portion (I) of the inventive fusion protein may either reflect the retro-inverso D-form of the native L-form analog or may reflect a derivative of the retro-inverso D-form (an analog of the native L-form) by introduction of sequence modification(s).

Derivatives of native D- or L-form sequences (forming inventive fusion proteins) typically have substantial identity with the native amino acid sequences, e.g. as disclosed herein by FIGS. 2A to 2F for the portion (II). Particularly preferred are amino acid sequences which have at least 60% sequence identity, preferably at least 75% sequence identity, more preferably at least 80%, still more preferably 90% sequence identity and most preferably at least 95% sequence identity thereto. Sequence identity can be measured, e.g. by using sequence analysis software (Sequence Analysis Software Package of the Genetics Computer Group, University of Wisconsin Biotechnology Center, 1710 University Avenue, Madison, Wis. 53705) with appropriate default parameters therein.

The production of native protein sequences composed of L-amino acids by recombinant techniques or by in vitro translation is well known and can be carried out following standard methods which are well known by a person skilled in the art (see e.g., Sambrook J, Maniatis T (1989) supra). Recombinant techniques may be used to prepare certain portions of the inventive fusion protein, e.g. portion (II), if it comprises native or derivative L-form sequences according to one embodiment of the present invention. These L-form portion (II) is then linked to the D-form portion (I) by a separate step as disclosed above. In general, the recombinant preparation of a portion of a fusion protein of the invention is carried out by either selecting the desired native DNA sequence or by modifying a native DNA sequence, by transformation of that DNA sequence into a suitable host and expression of the (modified) DNA sequence to form the desired protein sequence. The isolation of desired protein sequence can be carried out using standard methods including separating the (host) cells from the medium by centrifugation or filtration, if necessary after disruption of the cells, precipitating the proteineous components of the supernatant or filtrate by means of a salt, e.g., ammonium sulfate, followed by purification by using a variety of chromatographic procedures, e.g., ion exchange chromatography, affinity chromatography or similar art recognized procedures (see, e.g., Sambrook J, Maniatis T (1989) supra).

In more detail, the recombinant methods include transforming a suitable host cell with a nucleic acid or a vector provided herein which encodes the L-form of e.g. portion (II) of the fusion protein and cultivating the resultant recombinant microorganism, preferably E. coli, under conditions suitable for host cell growth and nucleic acid expression, e.g., in the presence of inducer, suitable media supplemented with appropriate salts, growth factors, antibiotic, nutritional supplements, etc.), whereby the nucleic acid is expressed and the encoded fusion protein is produced.

A vector comprising the nucleic acid of the native L-form of portion (II) of the fusion protein of the invention defines a nucleic acid sequence which comprises (apart from other sequences) one or more nucleic acid sequences of e.g. the L-form sequence of portion (II). A vector can be used, upon transformation into an appropriate host cell, to allow expression of said nucleic acid. The vector may be a plasmid, a phage particle or simply a potential genomic insert. Once transformed into a suitable host, the vector may replicate and function independently of the host genome, or may, under suitable conditions, integrate into the genome itself. Preferred vectors according to the invention are E. coli XL-Blue MRF′ and pBK-CMV plasmid.

The aforementioned term “other sequences” of a vector relate to the following: In general, a suitable vector includes an origin of replication, for example, Ori p, colEl Ori, sequences which allow the inserted nucleic acid to be expressed (transcribed and/or translated) and/or a selectable genetic marker including, e.g., a gene coding for a fluorescence protein, like GFP, genes which confer resistance to antibiotics such as the p-lactamase gene from Tn3, the kanamycin-resistance gene from Tn903 or the chloramphenicol-resistance gene from Tn9.

The term “plasmid” means an extrachromosomal usually self-replicating genetic element. Plasmids are generally designated by a lower “p” preceded and/or followed by letters and numbers. The starting plasmids herein are either commercially available, publicly available on an unrestricted basis or can be constructed from available plasmids in accordance with the published procedures. In addition, equivalent plasmids to those described are known to a person skilled in the art. The starting plasmid employed to prepare a vector of the present invention may be isolated, for example, from the appropriate E. coli containing these plasmids using standard procedures such as cesium chloride DNA isolation.

A suitable vector also relates to a (recombinant) DNA cloning vector as well as to a (recombinant) expression vector. A DNA cloning vector refers to an autonomously replicating agent, including, but not limited to, plasmids and phages, comprising a DNA molecule to which one or more additional nucleic acids of e.g portion (II) of the fusion protein in its native L-form have been added. An expression vector relates to any DNA cloning vector recombinant construct comprising a nucleic acid sequence to be expressed operably lined to a suitable control sequence capable of effecting the expression and to control the transcription of the inserted nucleic acid in a suitable host. Such plasmids may also be readily modified to construct expression vectors that produce the desired sequence in a variety of organisms, including, for example, E. coli, Sf9 (as host for baculovirus), Spodotera and Saccharomyces. The literature contains techniques for constructing AV12 expression vectors and for transforming AV12 host cells. U.S. Pat. No. 4,992,373, herein incorporated by reference, is one of many references describing these techniques.

“Operably linked” means that the nucleic acid sequence is linked to a control sequence in a manner which allows expression (e.g., transcription and/or translation) of the nucleic acid sequence. “Transcription” means the process whereby information contained in a nucleic acid sequence of DNA is transferred to complementary RNA sequence

“Control sequences” are well known in the art and are selected to express the nucleic acid of the L-form of the fusion protein of the invention and to control the transcription. Such control sequences include, but are not limited to a polyadenylation signal, a promoter (e.g., natural or synthetic promotor) or an enhancer to effect transcription, an optional operator sequence to control transcription, a locus control region or a silencer to allow a tissue-specific transcription, a sequence encoding suitable ribosome-binding sites on the mRNA, a sequence capable to stabilize the mRNA and sequences that control termination of transcription and translation. These control sequences can be modified, e.g., by deletion, addition, insertion or substitution of one or more nucleic acids, whereas saving their control function. Other suitable control sequences are well known in the art and are described, for example, in Goeddel (1990), Gene Expression Technology-Methods in Enzymology 185, Academic Press, San Diego, Calif.

Especially a high number of different promoters for different organism is known. For example, a preferred promoter for vectors used in Bacillus subtilis is the AprE promoter, a preferred promoter used in E. coli is the 17/Lac promoter, a preferred promoter used in Saccharomyces cerevisiae is PGK1, a preferred promoter used in Aspergillus niger is glaA, and a preferred promoter used in Trichoderma reesei (reesei) is cbhI. Promoters suitable for use with prokaryotic hosts also include the beta-lactamase (vector pGX2907 (ATCC 39344) containing the replicon and beta-lactamase gene) and lactose promoter systems (Chang et al. (1978), Nature (London), 275:615; Goeddel et al. (1979), Nature (London), 281:544), alkaline phosphatase, the tryptophan (trp) promoter system (vector pATH1 (ATCC 37695) designed to facilitate expression of an open reading frame as a trpE fusion protein under control of the trp promoter) and hybrid promoters such as the tac promoter (isolatable from plasmid pDR540 ATCC-37282). However, other functional bacterial promoters, whose nucleotide sequences are generally known, enable a person skilled in the art to ligate them to DNA encoding the desired protein sequence, e.g. portion (II) of the inventive fusion protein in its L-forms, using linkers or adapters to supply any required restriction sites. Promoters for use in bacterial systems also will contain a Shine-Dalgarno sequence operably linked to the DNA encoding the desired protein sequence to be used as (part of) the inventive fusion protein.

Useful expression vectors, for example, may consist of segments of chromosomal, non-chromosomal and synthetic DNA sequences such as various known derivatives or fragments of SV40 and known bacterial plasmids, e.g., plasmids from E. coli including col E1, pBK, pCR1, pBR322, pMb9, pUC 19 and their derivatives, wider host range plasmids, e.g., RP4, phage DNAs e.g., the numerous derivatives of phage lambda, e.g., NM989, and other DNA phages, e.g., M13 and filamentous single stranded DNA phages, yeast plasmids, vectors useful in eukaryotic cells, such as vectors useful in animal cells and vectors derived from combinations of plasmids and phage DNAs, such as plasmids which have been modified to employ phage DNA or other expression control sequences. Expression techniques using the expression vectors described above are known in the art and are described generally in, for example, Sambrook J, Maniatis T (1989) supra.

Suitable “cells” or “host cells” comprising an aforementioned vector or a nucleic acid of the e.g. portion (II) L-form of the fusion protein of the invention may serve as a host and expression vehicle for the sequence to be expressed. The host cell can be e.g., a prokaryotic, an eukaryotic or an archaeon cell. Host cells comprise (for example, as a result of transformation, transfection or transduction) a vector or nucleic acid as described herein include, but are not limited to, bacterial cells (e.g., R. marinus, E. coli, Streptomyces, Pseudomonas, Bacillus, Serratia marcescens, Salmonella typhimurium), fungi including yeasts (e.g., Saccharomyces cerevisiae, Pichia pastoris) and molds (e.g., Aspergillus sp.), insect cells (e.g., Sf9) or mammalian cells (e.g., COS, CHO). Preferably, host cells means the cells of E. coli. In general, a host cell may be selected modulating the expression of inserted sequences of interest or modifying or processing expressed proteins encoded by the sequences in the specific manner desired. Appropriate cells or cell lines or host systems may thus be chosen to ensure the desired modification and processing of the foreign protein is achieved. For example, protein expression within a bacterial system can be used to produce an unglycosylated core protein, whereas expression within mammalian cells ensures “native” glycosylation of a heterologous protein.

Eukaryotic host cells are not limited to use in a particular eukaryotic host cell. A variety of eukaryotic host cells are available, e.g., from depositories such as the American Type Culture Collection (ATCC) and are suitable for use with vectors as described above. The choice of a particular host cell depends on the character of the expression vector used. Eukaryotic host cells include mammalian cells as well as yeast cells. The imperfect fungus Saccharomyces cerevisiae is the most commonly used eukaryotic microorganism, although a number of other strains are commonly available. For expression in Saccharomyces sp., the plasmid YRp7 (ATCC-40053), for example, is commonly used (see. e.g., Stinchcomb L. et al. (1979) Nature, 282:39; Kingsman J. al. (1979), Gene, 7:141; S. Tschemper et al. (1980), Gene, 10:157). This plasmid already contains the trp gene which provides a selectable marker for a mutant strain of yeast lacking the ability to grow in tryptophan.

Suitable promoting sequences for use with yeast hosts include the promoters for 3-phosphoglycerate kinase (found on plasmid pAP12BD (ATCC 53231) and described in U.S. Pat. No. 4,935,350, issued Jun. 19, 1990, herein incorporated by reference) or other glycolytic enzymes such as enolase (found on plasmid pAC1 (ATCC 39532)), glyceraldehyde-3-phosphate dehydrogenase (derived from plasmid pHcGAPC1 (ATCC 57090, 57091)), hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase, as well as the alcohol dehydrogenase and pyruvate decarboxylase genes of Zymomonas mobills (U.S. Pat. No. 5,000,000 issued Mar. 19, 1991, herein incorporated by reference). Other yeast promoters, which are inducible promoters, having the additional advantage of their transcription being controllable by varying growth conditions, are the promoter regions for alcohol dehydrogenase 2, isocytochrome C, acid phosphatase, degradative enzymes associated with nitrogen metabolism, metallothionein (contained on plasmid vector pCL28XhoLHBPV (ATCC 39475) and described in U.S. Pat. No. 4,840,896, herein incorporated by reference), glyceraldehyde 3-phosphate dehydrogenase, and enzymes responsible for maltose and galactose (e.g. GAL1 found on plasmid pRY121 (ATCC 37658)) utilization. Yeast enhancers such as the UAS Ga1 from Saccharomyces cerevisiae (found in conjunction with the CYC1 promoter on plasmid YEpsec-hI1beta ATCC 67024), also are advantageously used with yeast promoters.

An aforementioned vector can be introduced into a host cell using any suitable method (e.g., transformation, electroporation, transfection using calcium chloride, rubidium chloride, calcium phosphate, DEAEdextran or other substances, microprojectile bombardment, lipofection, infection or transduction). Transformation relates to the introduction of DNA into an organism so that the DNA is replicable, either as an extrachromosomal element or by chromosomal integration. Methods of transforming bacterial and eukaryotic hosts are well known in the art. Numerous methods, such as nuclear injection, protoplast fusion or by calcium treatment are summerized in Sambrook J, Maniatis T (1989) supra. Transfection refers to the taking up of a vector by a host cell whether or not any coding sequences are in fact expressed. Successful transfection is generally recognized when any indication or the operation or this vector occurs within the host cell.

In another embodiment of the present invention, portion (II) of the inventive fusion protein does not correspond to the native L-form or a derivative thereof, but to a retro-inverso D-form sequence composed of D-amino acids and reversed in its order. These D-form sequences of portion (II) (as with the D-form sequence of portion (I)) are typically not amenable by recombinant techniques and are preferably synthesized by peptide synthesis technology, e.g. solid phase synthesis, using D-amino acids instead of L-amino acids to obtain a D-amino acid sequence. However, it has to be noted that the amino acid order has to be reversed for the synthesis (as compared to the L-form analog).

Correspondingly, all amino acid sequences of the invention (either D- or L-form sequences) can be constructed by chemical methods well known in the art, including solid phase protein synthesis, or recombinant methods. Both methods are described in U.S. Pat. No. 4,617,149, the entirety of which is herein incorporated by reference. The principles of solid phase chemical synthesis of fusion proteins are well known in the art and are described by, e.g., Dugas H and Penney C. (1981), Bioorganic Chemistry, pages 54-92. For examples, proteins and peptides may be synthesized by solid-phase methodology utilizing an Applied Biosystems 430A peptide synthesizer (commercially available from Applied Biosystems, Foster City, Calif.) and synthesis cycles supplied by Applied Biosystems. Protected amino acids, such as t-butoxycarbonyl-protected amino acids, and other reagents are commercially available from many chemical supply houses. Sequential t-butoxycarbonyl chemistry using double couple protocols are applied to the starting p-methyl benzhydryl amine resins for the production of C-terminal carboxamides. For the production of C-terminal acids, the corresponding pyridine-2-aldoxime methiodide resin is used. Asparagine, glutamine, and arginine are coupled using preformed hydroxy benzotriazole esters. The following side chain protection may be used:

Arg, Tosyl; Asp, cyclohexyl; Glu, cyclohexyl; Ser, Benzyl; Thr, Benzyl; Tyr, 4-bromo carbobenzoxy.

Removal of the t-butoxycarbonyl moiety (deprotection) may be accomplished with trifluoroacetic acid (TFA) in methylene chloride. Following completion of the synthesis the proteins or peptides may be deprotected and cleaved from the resin with anhydrous hydrogen fluoride containing 10% meta-cresol. Cleavage of the side chain protecting group(s) and of the (fusion) protein from the resin is carried out at zero degrees centigrade or below, preferably −20° C. for thirty minutes followed by thirty minutes at 0° C. After removal of the hydrogen fluoride, the (fusion) protein/resin is washed with ether, and the (fusion) protein extracted with glacial acetic acid and then lyophilized. Purification is accomplished by size-exclusion chromatography on a Sephadex G-10 (Pharmacia) column in 10% acetic acid. As explained above peptides composed of L-amino acids or D-amino acids are synthesized according to the same general technology.

Another embodiment of the present invention provides a pharmaceutical composition which comprises a fusion protein of the invention and optionally a pharmaceutically acceptable carrier, adjuvant and/or vehicle. This pharmaceutical composition is intended for the treatment of diseases which are at least in part caused by changes (e.g. mutations) in normal programmed cell death (apoptosis). In particular, such changes prevent (all or in part) normal programmed cell death (apoptosis). Preferably, these diseases include cancer, e.g., Hodgkin lymphoma, non-Hodgkin lymphoma, histocytic lymphoma, cancers of the brain (glioblastomas), ovarian, genitourinary tract, colon, liver, colorectal tract, pancreas, breast, prostate, lymphatic system, stomach, larynx and lung, including lung adenocarcinoma and small cell lung cancer and/or skin cancer, e.g. melanoma or non-melanoma skin cancer, including basal cell and squamous cell carcinomas as well as psoriasis, Behcet's syndrome, pemphigus vulgaris.

A “pharmaceutically acceptable carrier, adjuvant, or vehicle” according to the invention refers to a non-toxic carrier, adjuvant or vehicle that does not destroy the pharmacological activity of the fusion protein with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, polyethylene glycol and wool fat.

The pharmaceutical composition of the present invention may be administered parenterally or non-parenterally (e.g. orally).

The term “parenteral” as used herein includes subcutaneous, intravenous, intramuscular, intra-articular, intra-synovial, intrasternal, intrathecal, intrahepatic, intralesional and intracranial injection or infusion techniques. Preferably, the pharmaceutical compositions are administered orally, intraperitoneally or intravenously. Sterile injectable forms of the pharmaceutical compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

If administered orally, the pharmaceutical compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavouring or colouring agents may also be added. Additionally, standard pharmaceutical methods can be employed to control the duration of action. These are well known in the art and include control release preparations and can include appropriate macromolecules, for example polymers, polyesters, polyaminoacids, polyvinylpyrrolidone, ethylenevinylacetate, methyl cellulose, carboxymethyl cellulose or protamine sulfate. The concentration of macromolecules as well as a the methods of incorporation can be adjusted in order to control release. Additionally, the agent can be incorporated into particles of polymeric materials such as polyesters, polyaminoacids, hydrogels, poly(lactic acid) or ethylenevinylacetate copolymers. In addition to being incorporated, these agents can also be used to trap the compound in microcapsules.

Further administration forms are e.g. by inhalation spray, topically, rectally, nasally, bucally, vaginally, or via an implanted reservoir, some of which are described in the following in more detail.

Accordingly, the pharmaceutical compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract can be effected in a rectal suppository formulation (see above) or in a suitable enema formulation. Topically-transdermal patches may also be used. For topical applications, the pharmaceutical compositions may be formulated in a suitable ointment containing the active component suspended or dissolved in one or more carriers. Carriers for topical administration of the fusion proteins of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active fusion proteins suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride.

Alternatively, for ophthalmic uses, the pharmaceutical compositions may be formulated in an ointment such as petrolatum.

The pharmaceutical compositions of this invention may also be administered by nasal aerosol or inhalation. Such pharmaceutical compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, the pharmaceutical compositions of this invention are formulated for oral administration.

The amount of the fusion protein(s) of the present invention that may be combined with carriers, adjuvants and vehicles to produce a pharmaceutical composition in a single dosage form will vary depending upon the host treated, the particular mode of administration. Preferably, the pharmaceutical compositions should be formulated so that a dosage of between 0.01-100 mg/kg body weight/day of the inhibitor can be administered to a patient receiving these compositions. Preferred dosages range from 0.1-5 mg/kg body weight/day, even further preferred dosages from 1-5 mg/kg body weight/day.

Capsules: Capsules are prepared by filling standard two-piece hard gelatin capsulates each with 100 milligram of powdered active ingredient, 175 milligrams of lactose, 24 milligrams of talc and 6 milligrams magnesium stearate. Soft Gelatin Capsules: A mixture of active ingredient in soybean oil is prepared and injected by means of a positive displacement pump into gelatin to form soft gelatin capsules containing 100 milligrams of the active ingredient. The capsules are then washed and dried. Tablets: Tablets are prepared by conventional procedures so that the dosage unit is 100 milligrams of active ingredient. 0.2 milligrams of colloidal silicon dioxide, 5 milligrams of magnesium stearate, 275 milligrams of microcrystalline cellulose, 11 milligrams of cornstarch and 98.8 milligrams of lactose. Appropriate coatings may be applied to increase palatability or to delay absorption. Injectable: A parenteral composition suitable for administration by injection is prepared by stirring 1.5% by weight of active ingredients in 10% by volume propylene glycol and water. The solution is made isotonic with sodium chloride and sterilized. Suspension: An aqueous suspension is prepared for oral administration so that each 5 millimeters contain 100 milligrams of finely divided active ingredient, 200 milligrams of sodium carboxymethyl cellulose, 5 milligrams of sodium benzoate, 1.0 grams of sorbitol solution U.S.P. and 0.025 millimeters of vanillin.

It has to be noted that a specific dosage and treatment regimen for any particular patient will depend upon a variety of factors, including the activity of the specific fusion protein employed, the age, body weight, general health, sex, diet, time of administration, rate of excretion, drug combination, and the judgment of the treating physician and the severity of the particular disease being treated. The amount of a fusion protein of the present invention in the pharmaceutical composition will also depend upon the particular fusion protein in the composition.

A further embodiment of the invention relates to the use of a fusion protein of the invention for the treatment or for the preparation of a pharmaceutical composition for the treatment and/or prophylaxis of diseases or to therapeutic methods for the treatment of conditions caused by defective apoptosis, cancer, e.g., Hodgkin lymphoma, non-Hodgkin lymphoma, histocytic lymphoma, cancers of the brain (glioblastomas), ovarian, genitourinary tract, colon, liver, colorectal tract, pancreas, breast, prostate, lymphatic system, stomach, larynx and lung, including lung adenocarcinoma and small cell lung cancer and/or skin cancer, e.g. melanoma or non-melanoma skin cancer, including basal cell and squamous cell carcinomas as well as psoriasis, Behcet's syndrome, pemphigus vulgaris.

The following figures and examples are thought to illustrate the invention and should not be constructed to limit the scope of the invention thereon. All references cited by the disclosure of the present application are hereby incorporated in their entirety by reference.

### FIGURES

FIG. 1A shows the amino acid sequence of the basic region of the Tat protein in its retro-inverso D-form. Therefore, all amino acids shown are D-enantiomeric amino acids. The sequence of FIG. 1A is an examples for a D-TAT (SEQ ID NO:1).

FIG. 1B shows the amino acid sequence of generic D-TAT (SEQ ID NO:2). It is referred to the description of FIG. 1A.

FIG. 2A shows the native L-amino acid sequence of Bid (transcript variant 1, SEQ ID NO:3), human isoform.

FIG. 2B shows the native L-amino acid sequence of Bad (SEQ ID NO:4); human isoform; the BH3 domain is underlined. The BH3 domain can comprise the underlined section but also two additional amino acids at either terminus. These four additional amino acids are indicated in bold letters.

FIG. 2C shows the native L-amino acid sequence of Noxa (SEQ ID NO:5), human isoform.

FIG. 2D shows the native L-amino acid sequence of Puma (SEQ ID NO:6), human isoform.

FIG. 2E shows the native L-amino acid sequence of Bim (transcript variant 1, SEQ ID NO:7), human isoform.

FIG. 2F shows the native L-amino acid sequence of Bik (SEQ ID NO:8); human isoform; the BH3 domain is underlined. The BH3 domain can comprise the underlined section but also two additional amino acids at either terminus. These four additional amino acids are indicated in bold letters.

FIG. 3A shows the amino acid sequence of D-TAT-Bid (transcript variant 1, SEQ ID NO:9)—an example of an inventive fusion protein, whereby the Bid full-length sequence (according to the invention, portion (II) of the inventive fusion protein) is depicted in its native L-form (as with the BH3-only protein sequences of the following FIG. 3B to 3F) and portion (I) in its inverted form composed of D-amino acids (D-Tat). In order to depict the inventive fusion protein as retro-inverso all-D-amino acid sequence, portion (II) has to be inverted.

FIG. 3B shows the amino acid sequence of D-TAT-Bad (SEQ ID NO:10); the BH3 domain is underlined. The BH3 domain can comprise the underlined section but also two additional amino acids at either terminus. These four additional amino acids are indicated in bold letters.

FIG. 3C shows the amino acid sequence of D-TAT-Noxa (SEQ ID NO:11).

FIG. 3D shows the amino acid sequence of D-TAT-Puma (SEQ ID NO:12).

FIG. 3E shows the amino acid sequence of D-TAT-Bim (transcript variant 1, (SEQ ID NO:13).

FIG. 3F shows the amino acid sequence of D-TAT-Bik (SEQ ID NO:14); the BH3 domain is underlined. The BH3 domain can comprise the underlined section but also two additional amino acids at either terminus. These four additional amino acids are indicated in bold letters.

FIG. 4A shows the native L-amino acid sequence of the BH3-domain of Bik (Bik BH3);

FIG. 4B shows the native L-amino acid sequence of the BH3-domain of Bad (Bad BH3);

FIG. 4C shows the native L-amino acid sequence of the BH3-domain of Bid (Bid BH3);

FIG. 4D shows the native L-amino acid sequence of the BH3-domain of Bmf (Bmf BH3);

FIG. 4E shows the native L-amino acid sequence of the BH3-domain of DP5/Hrk (DP5/Hrk BH3);

FIG. 4F shows the native L-amino acid sequence of the BH3-domain of Bim (Bim BH3);

FIG. 4G shows the native L-amino acid sequence of the BH3-domain of Noxa (Noxa BH3);

FIG. 4H shows the native L-amino acid sequence of the BH3-domain of PUMA (PUMA BH3);

FIG. 4I shows the native L-amino acid sequence of the BH3-domain of Bax (Bax BH3);

FIG. 4J shows the native L-amino acid sequence of the BH3-domain of Bak (Bak BH3);

FIG. 4K shows the native L-amino acid sequence of the BH3-domain of Bok (Bok BH3);

FIG. 5 shows a graph representing the results of BH3-induced cell death of HeLa cells. Used inventive fusion peptides comprise as first portion (I) the trafficking sequence D-TAT and as second portion (II) the BH3-domain sequences of BH3-only proteins Bim, Bok, Bax, DP5 [Hrk], Bid, Noxa, PUMA and Bak, respectively (as indicated in FIG. 5). Fusion peptides were incubated at concentrations of 1 μM, 10 μM, 50 μM and 100 μM with HeLa cells in culture medium for 24 hours. The control contains HeLa cells without treatment, i.e., no incubation with inventive fusion peptides. The control was also incubated with βTC-3 cells in culture medium for 24 hours. Apoptotic cells were counted following Hoechst/PI staining (Bonny et al., J. Biol. Chem. (2000) 275:16466-16472). The percentage rate of apoptotic cells is indicated in FIG. 5 on the axis of abscissae as “% cell death”. As can be seen in FIG. 5, fusion peptides comprising Bim, Bok or Bax show the best results, i.e. the highest cell death rates of about 98% and 68%, followed by the fusion peptide comprising PUMA (cell death rate of about 23%). It has to be noted that the fusion peptide comprising Bim leads to the best result, i.e. a cell death rate of about 98% at a peptide concentration of 50 μM, whereas fusion peptides comprising Bok or Bax lead to indicated cell death rates of about 98% and about 68% at a peptide concentration of 100 μM.

FIG. 6 shows a graph representing the results of BH3-induced cell death of βTC-3 cells. Used inventive fusion peptides comprise as first portion (I) the trafficking sequence D-TAT and as second portion (II) the BH3-domain sequences of BH3-only proteins Puma, Noxa, Bak Bim, Bok, Bax and DP-5, respectively (as indicated in FIG. 6). Fusion peptides were incubated at concentrations of 1 μM and 10 μM, respectively, (indicated as “Puma 1” for concentration of 1 μM and “Puma 10” for concentration 10 μM and so forth) with βTC-3 cells in culture medium for 24 hours. One sample contains the trafficking sequence L-Tat at a concentration of 10 μM. Another sample contains the substance thapsigargin as toxicity control. The control contains βTC-3 cells without treatment, i.e., no incubation with inventive fusion peptides. The L-Tat sample and the control were also incubated with βTC-3 cells in culture medium for 24 hours. Apoptotic cells were counted following Hoechst/PI staining (see above). The percentage rate of apoptotic cells is indicated in FIG. 6 on the axis of abscissae as “% cell death”. As can be seen in FIG. 6, fusion peptides comprising Bim, Bok at concentrations of 10 μM show the best results (cell death of about 99% and about 86%, respectively followed by Noxa and Bax at concentrations of 10 μM (cell death of about 52% and about 43%, respectively).

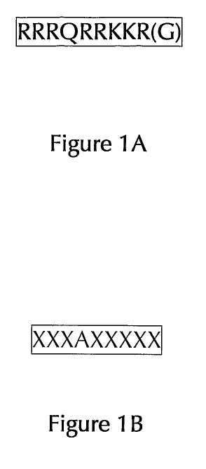

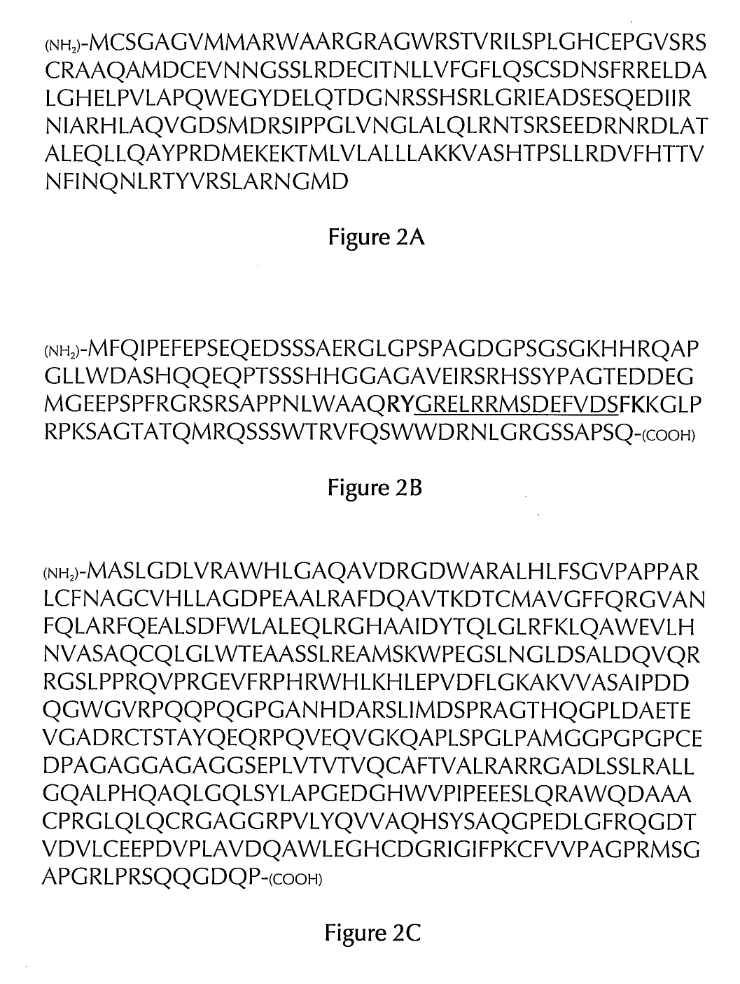

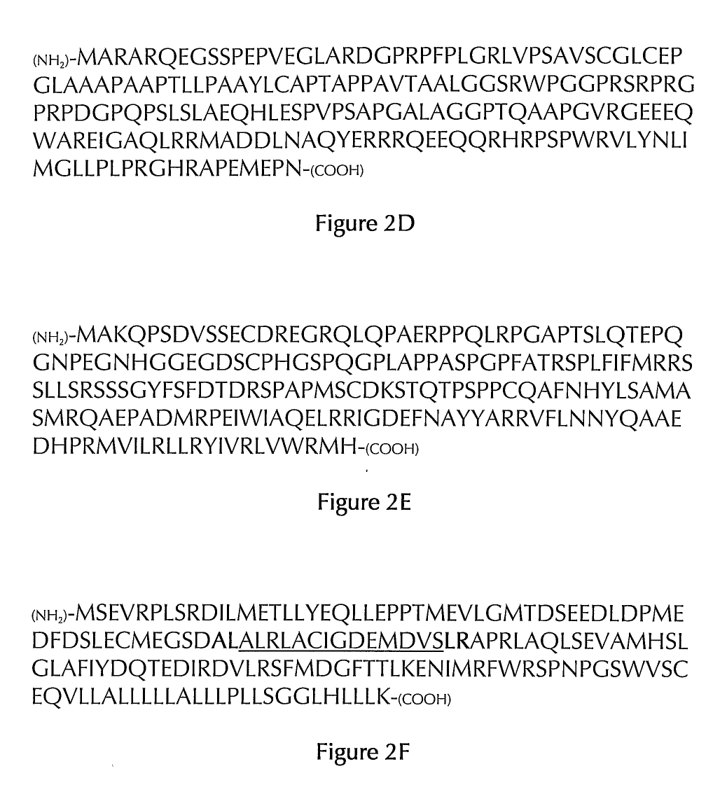

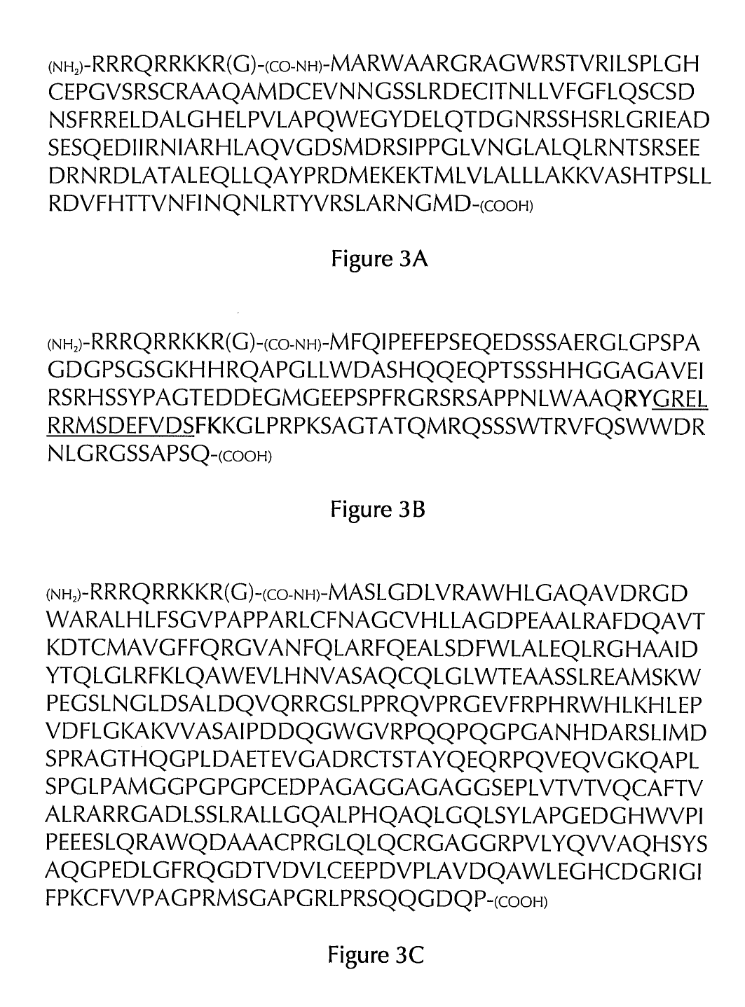

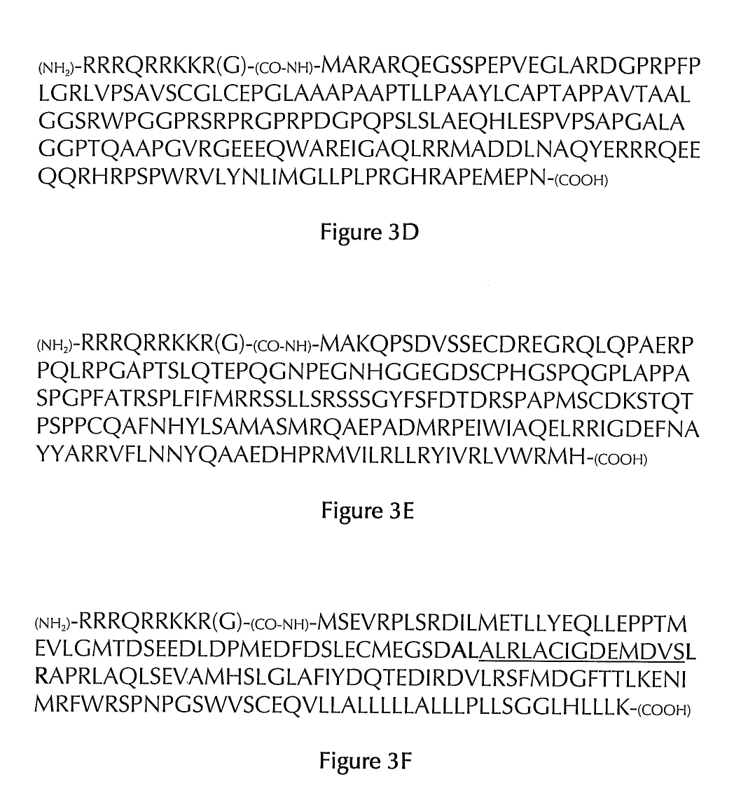

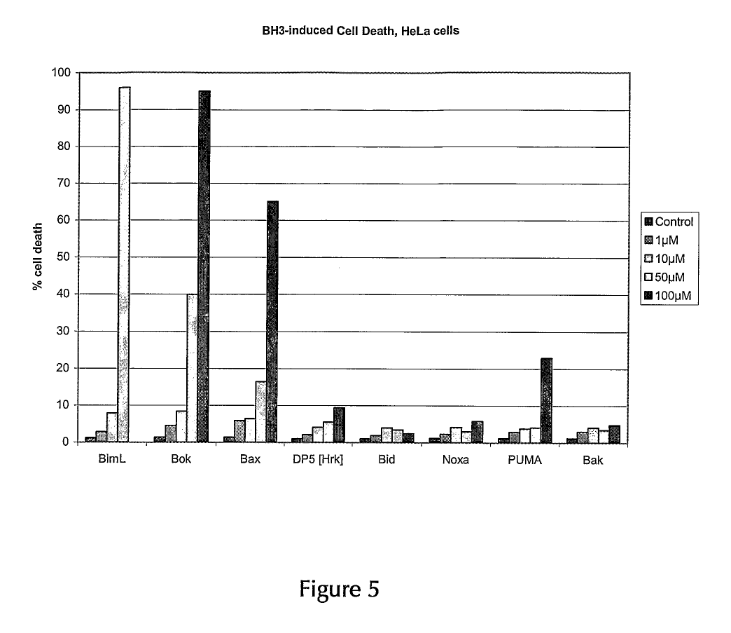

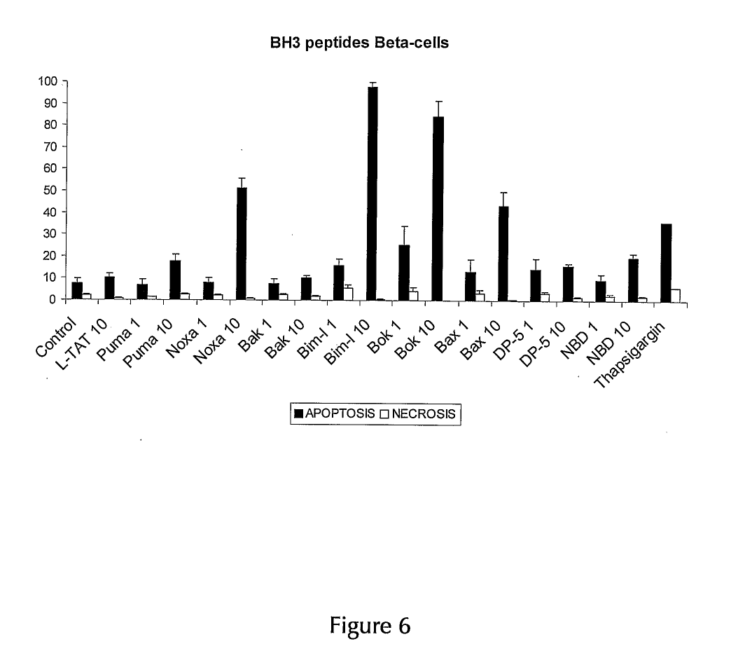

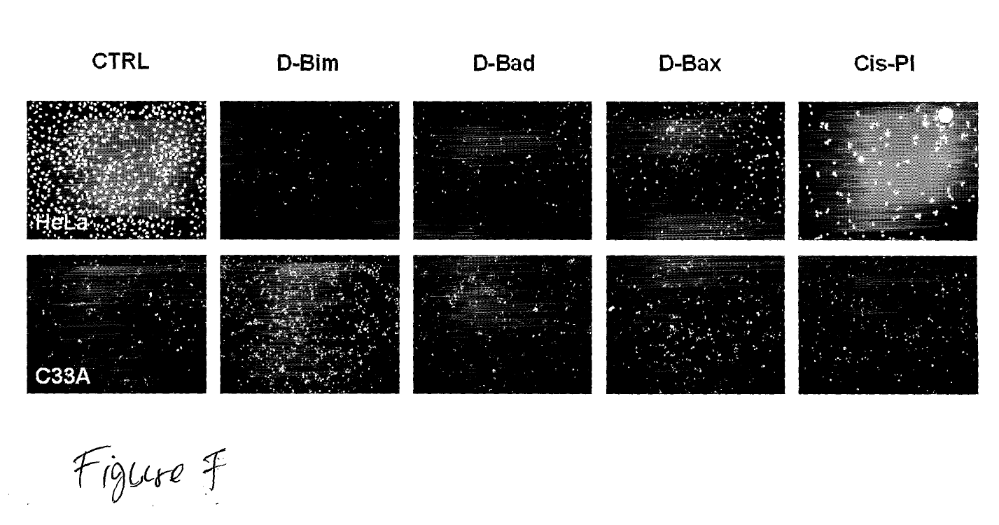

The Hoechst/PI staining as indicated in FIGS. 5 and 6 was performed as described in Bonny C. et al., IB1 reduces cytokine-induced apoptosis of insulin-secreting cells, J. Biol. Chem. (2000) 275:16466-16472. Both cell lines, the HeLa cell-line (Human cervix cancer) (FIG. 5) and insulin-secreting cell line βTC-3 (Efrat S. et al., Beta-cell lines derived from transgenic mice expressing a hybrid insulin gene-oncogene, Proc Natl Acad Sci USA (1988) 85: 9037-9041) (FIG. 6) were cultured in RPMI-1640 medium supplemented with 10% fetal calf serum, 100 μg/ml streptomycin, 100 U/ml penicillin, 1 mmol/l Na-pyruvate, and 2 mmol/l glutamine.

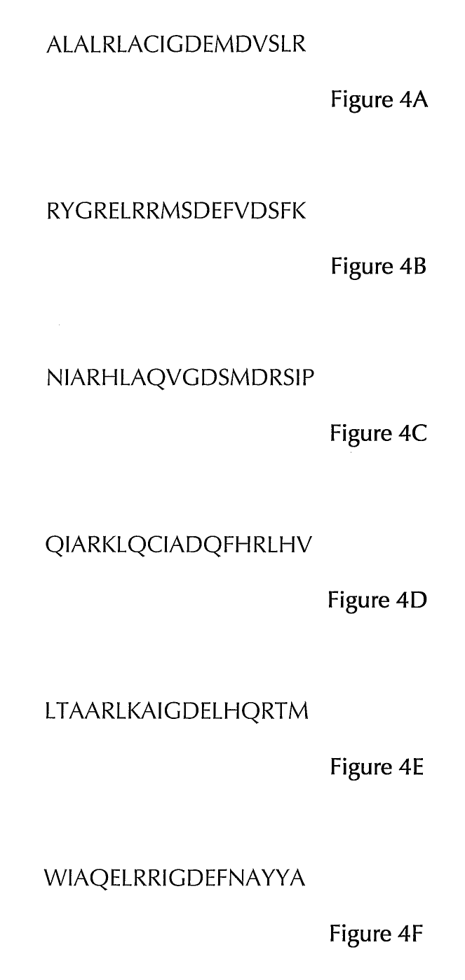

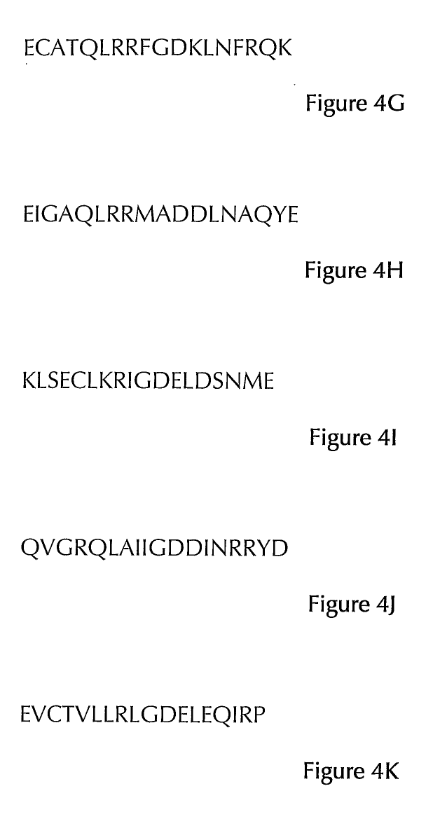

FIG. 7 shows the results obtained for inventive all-D fusion peptides (D-BH3-D-Tat from Bid, Bax and Bad). The BH3-domain sequences composed of D amino acids correspond to the sequences given in FIG. 4, however, with the amino acid sequence inverted. All concentrations used including Cis-Pl were at 10 μM. Incubation of cells (HeLa (upper row) and C33A (lower row)) lasted for 48 hours. The following conditions were applied: RPMI-1640 medium supplemented with 10% fetal calf serum, 100 μg/ml streptomycin, 100 U/ml penicillin, 1 mmol/l Na-pyruvate, and 2 mmol/l glutamine was prepared. Staining was carried out with Hoechst and Propidium Iodide (PI) (see above) (1 μg/ml each for 5 minutes, then manual counting of cells under a fluorescent microscope or take picture). Viable cells stain blue, dead ones stain dark red (dark) in this assay. It is clearly visible that all inventive all-D fusion peptides render the cells apoptotic.

1. A fusion protein comprising at least one first portion (I) comprising a trafficking sequence and at least one second portion (II) comprising a partial or full-length BH3-domain sequence of a BH3-only protein, said fusion protein comprising D-enantiomeric amino acids in its portion (I) in retro-inverso order.

2. The fusion protein of claim 1, wherein the at least one first portion (I) and the at least one second portion (II) are linked by a covalent bond.

3. The fusion protein of claim 1, wherein portion (I) is capable of directing the fusion protein to a defined cellular location.

4. The fusion protein of claim 1, wherein portion (I) is capable of enhancing cell permeability.

5. The fusion protein of claim 1, wherein portion (I) comprises the TAT protein of a human immunodeficiency virus.

6. The fusion protein of claim 1, wherein portion (I) comprises the amino acid sequence of SEQ ID NO:1 or SEQ ID NO:2.

7. The fusion protein of claim 1, wherein portion (II) comprises a partial or full-length BH3-domain sequence that induces apoptosis.

8. The fusion protein of claim 1, wherein portion (II) comprises a partial or full-length BH3-domain sequence that interacts with at least one Bcl-2 family protein.

9. The fusion protein of claim 1, wherein portion (II) comprises a partial or full-length BH3-domain sequence that either activates or sensitizes at least one pro-apoptotic member of the Bcl-2 family.

10. The fusion protein of claim 1, wherein portion (II) comprises a partial or full-length BH3-domain sequence from a BH3-only protein selected from the group consisting of Bid, Bad, Noxa, Puma, Bim, Bik, Bmf, DP5/Hrk and Bok.

11. The fusion protein of claim 1, wherein portion (II) comprises a partial or full-length BH3-domain sequence in a form selected from the group consisting of its native form comprising L-amino acids and its inverted form comprising D-amino acids.

12. The fusion protein of claim 1, wherein portion (II) comprises the amino acid sequence selected from the group consisting of SEQ ID NO:3 SEQ ID NO:4 SEQ ID NO:5 SEQ ID NO:6 SEQ ID NO:7 SEQ ID NO:8 and fragments thereof.

13. A pharmaceutical composition comprising a fusion protein of claim 1 and a pharmaceutically acceptable carrier, adjuvant or vehicle.

14-15. (canceled)

16. A method of treating a disease comprising administrating the pharmaceutical compositions of claim 13 to a patient in need of treatment.

17. The method of claim 16, where said disease is selected from the group consisting of Hodgkin lymphoma, non-Hodgkin lymphoma, histocytic lymphoma, glioblastomas, ovarian cancer, genitourinary tract cancer, colon cancer, liver cancer, colorectal cancer, pancreatic cancer, breast cancer, prostate cancer, lymphatic system cancer, stomach cancer, larynx and lung cancer, lung adenocarcinoma, small cell lung cancer, melanoma and non-melanoma skin cancer, psoriasis, Behcet's syndrome and pemphigus vulgaris.


SEQUENCE LISTING (57 sequences)

SEQ ID NO: 1 - protein, 10 aa, Artificial Sequence
  Description of Artificial Sequence Synthetic peptide
RRRQRRKKRG

SEQ ID NO: 2 - protein, 9 aa, Artificial Sequence
  Description of Artificial Sequence Synthetic peptide
XXXXXXXXX

SEQ ID NO: 3 - protein, 241 aa, Homo sapiens
MCSGAGVMMARWAARGRAGWRSTVRILSPLGHCEPGVSRSCRAAQAMDCEVNNGSSLRDECITNLLVFGFLQSCSDNSFRRELDALGHELPVLAPQWEGYDELQTDGNRSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 4 - protein, 168 aa, Homo sapiens
MFQIPEFEPSEQEDSSSAERGLGPSPAGDGPSGSGKHHRQAPGLLWDASHQQEQPTSSSHHGGAGAVEIRSRHSSYPAGTEDDEGMGEEPSPFRGRSRSAPPNLWAAQRYGRELRRMSDEFVDSFKKGLPRPKSAGTATQMRQSSSWTRVFQSWWDRNLGRGSSAPSQ

SEQ ID NO: 5 - protein, 483 aa, Homo sapiens
MASLGDLVRAWHLGAQAVDRGDWARALHLFSGVPAPPARLCFNAGCVHLLAGDPEAALRAFDQAVTKDTCMAVGFFQRGVANFQLARFQEALSDFWLALEQLRGHAAIDYTQLGLRFKLQAWEVLHNVASAQCQLGLWTEAASSLREAMSKWPEGSLNGLDSALDQVQRRGSLPPRQVPRGEVFRPHRWHLKHLEPVDFLGKAKVVASAIPDDQGWGVRPQQPQGPGANHDARSLIMDSPRAGTHQGPLDAETEVGADRCTSTAYQEQRPQVEQVGKQAPLSPGLPAMGGPGPGPCEDPAGAGGAGAGGSEPLVTVTVQCAFTVALRARRGADLSSLRALLGQALPHQAQLGQLSYLAPGEDGHWVPIPEEESLQRAWQDAAACPRGLQLQCRGAGGRPVLYQVVAQHSYSAQGPEDLGFRQGDTVDVLCEEPDVPLAVDQAWLEGHCDGRIGIFPKCFVVPAGPRMSGAPGRLPRSQQGDQP

SEQ ID NO: 6 - protein, 193 aa, Homo sapiens
MARARQEGSSPEPVEGLARDGPRPFPLGRLVPSAVSCGLCEPGLAAAPAAPTLLPAAYLCAPTAPPAVTAALGGSRWPGGPRSRPRGPRPDGPQPSLSLAEQHLESPVPSAPGALAGGPTQAAPGVRGEEEQWAREIGAQLRRMADDLNAQYERRRQEEQQRHRPSPWRVLYNLIMGLLPLPRGHRAPEMEPN

SEQ ID NO: 7 - protein, 198 aa, Homo sapiens
MAKQPSDVSSECDREGRQLQPAERPPQLRPGAPTSLQTEPQGNPEGNHGGEGDSCPHGSPQGPLAPPASPGPFATRSPLFIFMRRSSLLSRSSSGYFSFDTDRSPAPMSCDKSTQTPSPPCQAFNHYLSAMASMRQAEPADMRPEIWIAQELRRIGDEFNAYYARRVFLNNYQAAEDHPRMVILRLLRYIVRLVWRMH

SEQ ID NO: 8 - protein, 160 aa, Homo sapiens
MSEVRPLSRDILMETLLYEQLLEPPTMEVLGMTDSEEDLDPMEDFDSLECMEGSDALALRLACIGDEMDVSLRAPRLAQLSEVAMHSLGLAFIYDQTEDIRDVLRSFMDGFTTLKENIMRFWRSPNPGSWVSCEQVLLALLLLLALLLPLLSGGLHLLLK

SEQ ID NO: 9 - protein, 233 aa, Homo sapiens
MARWAARGRAGWRSTVRILSPLGHCEPGVSRSCRAAQAMDCEVNNGSSLRDECITNLLVFGFLQSCSDNSFRRELDALGHELPVLAPQWEGYDELQTDGNRSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 10 - protein, 168 aa, Homo sapiens; identical to SEQ ID NO: 4

SEQ ID NO: 11 - protein, 483 aa, Homo sapiens; identical to SEQ ID NO: 5

SEQ ID NO: 12 - protein, 193 aa, Homo sapiens; identical to SEQ ID NO: 6

SEQ ID NO: 13 - protein, 198 aa, Homo sapiens; identical to SEQ ID NO: 7

SEQ ID NO: 14 - protein, 160 aa, Homo sapiens; identical to SEQ ID NO: 8

SEQ ID NO: 15 - protein, 18 aa, Homo sapiens
ALALRLACIGDEMDVSLR

SEQ ID NO: 16 - protein, 18 aa, Homo sapiens
RYGRELRRMSDEFVDSFK

SEQ ID NO: 17 - protein, 18 aa, Homo sapiens
NIARHLAQVGDSMDRSIP

SEQ ID NO: 18 - protein, 18 aa, Homo sapiens
QIARKLQCIADQFHRLHV

SEQ ID NO: 19 - protein, 18 aa, Homo sapiens
LTAARLKAIGDELHQRTM

SEQ ID NO: 20 - protein, 18 aa, Homo sapiens
WIAQELRRIGDEFNAYYA

SEQ ID NO: 21 - protein, 18 aa, Homo sapiens
ECATQLRRFGDKLNFRQK

SEQ ID NO: 22 - protein, 18 aa, Homo sapiens
EIGAQLRRMADDLNAQYE

SEQ ID NO: 23 - protein, 18 aa, Homo sapiens
KLSECLKRIGDELDSNME

SEQ ID NO: 24 - protein, 18 aa, Homo sapiens
QVGRQLAIIGDDINRRYD

SEQ ID NO: 25 - protein, 18 aa, Homo sapiens
EVCTVLLRLGDELEQIRP

SEQ ID NO: 26 - protein, 10 aa, Artificial Sequence
  Description of Artificial Sequence Synthetic peptide
RRRQRRKKRG

SEQ ID NO: 27 - protein, 9 aa, Artificial Sequence
  Description of Artificial Sequence Synthetic peptide
RRRQRRKKR

SEQ ID NO: 28 - protein, 4 aa, Artificial Sequence
  Description of Artificial Sequence Synthetic peptide
KTRR

SEQ ID NO: 29 - protein, 4 aa, Artificial Sequence
  Description of Artificial Sequence Synthetic peptide
RLKR

SEQ ID NO: 30 - protein, 4 aa, Artificial Sequence
  Description of Artificial Sequence Synthetic peptide
KPRR

SEQ ID NO: 31 - protein, 5 aa, Artificial Sequence
  Description of Artificial Sequence Synthetic peptide
KRFQR

SEQ ID NO: 32 - protein, 5 aa, Artificial Sequence
  Description of Artificial Sequence Synthetic peptide
GRIRR

SEQ ID NO: 33 - protein, 7 aa, Artificial Sequence
  Description of Artificial Sequence Synthetic peptide
NIGRRRN

SEQ ID NO: 34 - protein, 7 aa, Artificial Sequence
  Description of Artificial Sequence Synthetic peptide
RAGRNGR

SEQ ID NO: 35 - protein, 4 aa, Artificial Sequence
  Description of Artificial Sequence Synthetic peptide
RPRR

SEQ ID NO: 36 - protein, 5 aa, Artificial Sequence
  Description of Artificial Sequence Synthetic peptide
GKRRG

SEQ ID NO: 37 - protein, 4 aa, Artificial Sequence
  Description of Artificial Sequence Synthetic peptide
KRRE

SEQ ID NO: 38 - protein, 7 aa, Artificial Sequence
  Description of Artificial Sequence Synthetic peptide
RQKRGGS

SEQ ID NO: 39 - protein, 4 aa, Artificial Sequence
  Description of Artificial Sequence Synthetic peptide
RKSR

SEQ ID NO: 40 - protein, 5 aa, Artificial Sequence
  Description of Artificial Sequence Synthetic peptide
RGSRR

SEQ ID NO: 41 - protein, 4 aa, Artificial Sequence
  Description of Artificial Sequence Synthetic peptide
RRQK

SEQ ID NO: 42 - protein, 5 aa, Artificial Sequence
  Description of Artificial Sequence Synthetic peptide
RARKG

SEQ ID NO: 43 - protein, 4 aa, Artificial Sequence
  Description of Artificial Sequence Synthetic peptide
RGRK

SEQ ID NO: 44 - protein, 5 aa, Artificial Sequence
  Description of Artificial Sequence Synthetic peptide
RRRLS

SEQ ID NO: 45 - protein, 7 aa, Artificial Sequence
  Description of Artificial Sequence Synthetic peptide
RPRRLSP

SEQ ID NO: 46 - protein, 5 aa, Artificial Sequence
  Description of Artificial Sequence Synthetic peptide
RGRKY

SEQ ID NO: 47 - protein, 7 aa, Artificial Sequence
  Description of Artificial Sequence Synthetic peptide
RPKRGMG

SEQ ID NO: 48 - protein, 5 aa, Artificial Sequence
  Description of Artificial Sequence Synthetic peptide
GVRRR

SEQ ID NO: 49 - protein, 9 aa, Artificial Sequence
  Description of Artificial Sequence Synthetic peptide
GYKKVGFSR

SEQ ID NO: 50 - protein, 7 aa, Artificial Sequence
  Description of Artificial Sequence Synthetic peptide
KFSRLSK

SEQ ID NO: 51 - protein, 4 aa, Artificial Sequence
  Description of Artificial Sequence Synthetic peptide
RRVR

SEQ ID NO: 52 - protein, 5 aa, Artificial Sequence
  Description of Artificial Sequence Synthetic peptide
RRSRP

SEQ ID NO: 53 - protein, 4 aa, Artificial Sequence
  Description of Artificial Sequence Synthetic peptide
RRRM

SEQ ID NO: 54 - protein, 11 aa, Artificial Sequence
  Description of Artificial Sequence Synthetic peptide
KSMALTRKGGY

SEQ ID NO: 55 - protein, 5 aa, Artificial Sequence
  Description of Artificial Sequence Synthetic peptide
RSRRG

SEQ ID NO: 56 - protein, 7 aa, Artificial Sequence
  Description of Artificial Sequence Synthetic peptide
KMNPLPY

SEQ ID NO: 57 - protein, 6 aa, Artificial Sequence
  Description of Artificial Sequence Synthetic 6xHis tag
HHHHHH